(19) United States
(12) Patent Application Publication
(10) Pub. No.: US 2003/0180395 A1
(43) Pub. Date: Sep. 25, 2003

(54) Plant extract

(76) Inventor: Bernd Bueter, Witterswil (CH)

Correspondence Address: BLANK ROME LLP, 600 NEW HAMPSHIRE AVENUE, N.W., WASHINGTON, DC, 20037, US

(21) Appl. No.: 10/333,627

(22) PCT Filed: Jul. 26, 2001
(86) PCT No.: PCT/CH01/00462

(30) Foreign Application Priority Data
Jul. 26, 2000 (CH) 1476/00

(51) Int. Cl. A61K 35/78

(52) U.S. Cl. 424/725

(57) ABSTRACT

The invention relates to an extract taken from Piper methysticum G. Forster, which is extracted from above-ground growing parts of these plants, especially from the leaves. Said extract offers advantages with regard to the action and extraction and, according to HPLC analysis, is distinctly different from known extracts taken from root material. One such extract can be obtained by extracting substances from above-ground growing plant material of Piper methysticum<G. Forster, preferably from the leaf material, and is suited for use in medicaments having an anxiolytic, anticonvulsive, muscle relaxant, narcosis increasing, analgesic, sleep-inducing, anti-inflammatory and/or neuroprotective effect.

The present invention relates to an extract of Piper methysticum G. Forster, which is different to the well-known extracts and offers benefits concerning its properties and production.

Extracts of Piper methysticum G. Forster are known, e.g. from WO 92/04036 and EP-A-0 987 026 and have anxiolytic, anticonvulsant, muscle relaxant, anaesthetic potentiating, analgesic, sleep inducing and neuroprotective properties. These effects have been ascribed to the presence of kavapyrones or kavalactones, in particularly kavain, 7,8-dihydrokavain, methysticin, 7,8-dihydromethysticin, yangonin, and 5,6-desmethoxyyangonin. It is assumed in this context that the kavapyrones are predominantly responsible for the pharmacological properties.

Pharmacological studies have been carried out using, on the one hand, synthetically produced kavapyrones or mixtures of synthetic kavapyrones and, on the other hand, plant extracts such as extensively described, by R. Hansel in “Kava-Kava in der modernen Arzneimittelforschung (Kava Kava in Modern Medicine Research)” Zeitschrift fur Phytotherapie journal for phytotherapy) (Hippokrates Verlag Stuttgart) 17 (1996), 180-195, where the chemical structures of the active components are disclosed as well.

The plant material used for production of these prior art plant extracts is root material or, respectively, the dried rootstock of Piper methysticum G. Forster (also termed kava-kava rhizome or kava rhizome), the roots adhering to the rhizome or the lateral roots, or a mix of rhizome parts, lateral roots and kava peelings (the partially rolled, externally grey-brown, internally yellow rootstock shavings). Suitable extraction procedures are described in the literature referred to above. The chemical structure of the main components of the extracts from the rootstock of the plants have been disclosed in Chimia 52 (1998) 443.

More recent studies have shown that damage to the liver may be caused by Kava preparations commercially available as medicaments or drugs which are obtained from root material of the Piper methysticum G. Forster and which for attaining of a pharmacological effect are standardized to a specific kavapyrone content in terms of the six kavapyrones mentioned above.

Further, using the rootstock, the root or root parts for the production of extracts for use in medicaments or remedies leads to the destruction of the plants.

It is an object of this invention to provide an extract of Piper methysticum G. Forster, which avoids or reduces the disadvantages associated with the well-known extracts.

Surprisingly, the investigations leading to the present invention indicated that extracts from the root material of Piper methysticum G. Forster were significantly different from the extracts obtained from plant material growing above ground, notably extracts obtained from the leaves as can be verified by chromatographic analysis.

A first embodiment of the extract according to the invention is defined in claim 1. Preferred embodiments of the extract according to the invention have the features specified in claims 2-5.

A further general embodiment of the extract according to the invention has the features specified in claim 6 while preferred embodiments have the features set forth in claim 7.

The invention further includes a process with the features set forth in claim 8.

Yet, the invention further concerns the use defined in claim 9 and the medicament defined in claim 10.

The term “extract” is understood to refer to a material which is obtained by extraction, maceration or percolation of a plant material with a suitable solvent and, optionally, by partial or complete removal of the solvent. Thus, extracts in accordance with this invention are either so-called dry extracts obtained by evaporation to dryness, or solvent-processed fluid extracts. Solvents suitable for extraction, percolation or the like are known to those experienced in the art. In particular, acetone, chloroform, ethyl acetate, lower alkanols with 1 to 4 C atoms, methanol and ethanol or a mixture of these and water are particularly suited. Carbon dioxide in fluid or super-critical form and pressurized gases with solvent properties are also suitable as extraction agents.

The term “above-ground growing plant material” of Piper methysticum G. Forster in the context of this invention is to be understood as referring to the fresh or dried material from the leaves and/or leaf stems, which may be harvested without significantly adversely affecting the growth capability of the plant, even when harvested in substantial volumes of 90% (typically 50%). This is advantageous in that cultures of the perennial plant can be repeatedly harvested without the planting of new plants being required. This, however, is not the only advantage of the inventive extract from above-ground growing plant material, notably the leaf material of Piper methysticum G. Forster.

It is understood that for the preparation of pharmaceutical preparations the extract may be processed together with conventional pharmaceutical adjuvants to yield capsules, (film)tablets, dragees, etc. Thus, it is apparent that fillers, binding agents, lubricants, and/or coating agents may be used as pharmaceutical adjuvants, e.g. for film-tablets and tablets and dragees.

Further, pharmaceutically preparations produced from an extract according to the invention may contain additional pharmaceutically active agents.

The extract according to the invention, or the medicaments produced there from have anxiolytic, anticonvulsive, muscle relaxant, anaesthetic potentiating, analgesic, sleep inducing, anti-inflammatory and/or neuroprotective effects.

The extract according to the invention can be used as such, or after further processing, e.g. to remove the coloring flavokavines. This may be done, e.g. by cold precipitation or by extraction in the presence of suitable adsorbents, such as particulate γ-aluminium oxide, or other conventional means.

In the appended drawings:

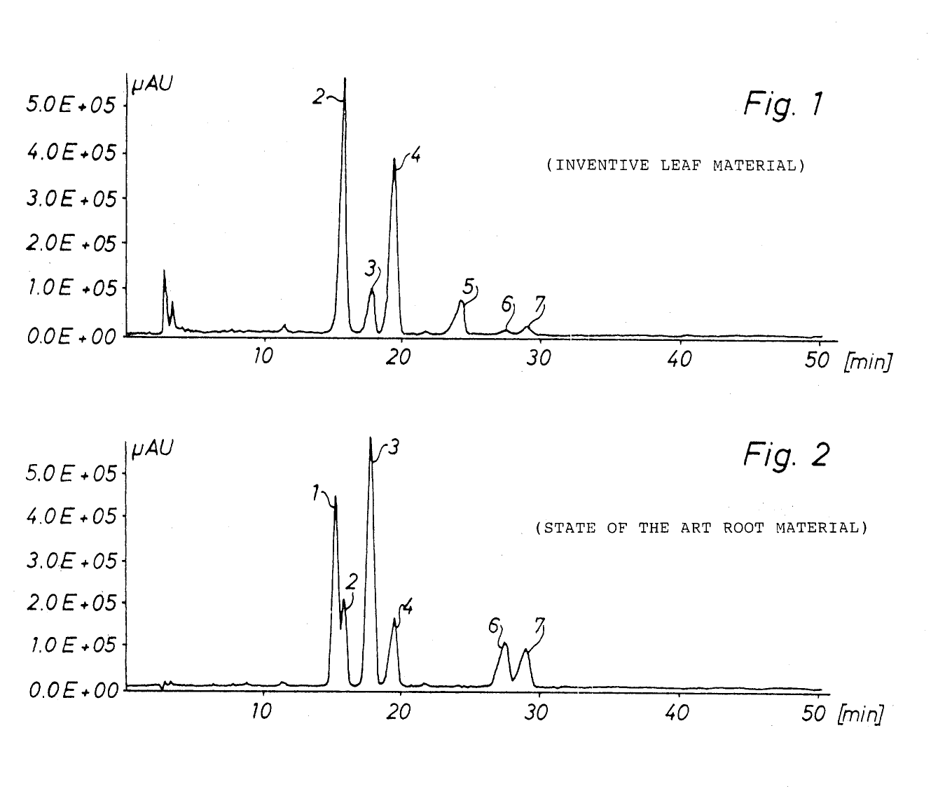

FIG. 1 shows the HPLC chromatogram of an inventive extract of Piper methysticum G. Forster from leaf material;

FIG. 2 shows an HPLC chromatogram of a prior art extract of Piper methysticum G. Forster from root material;

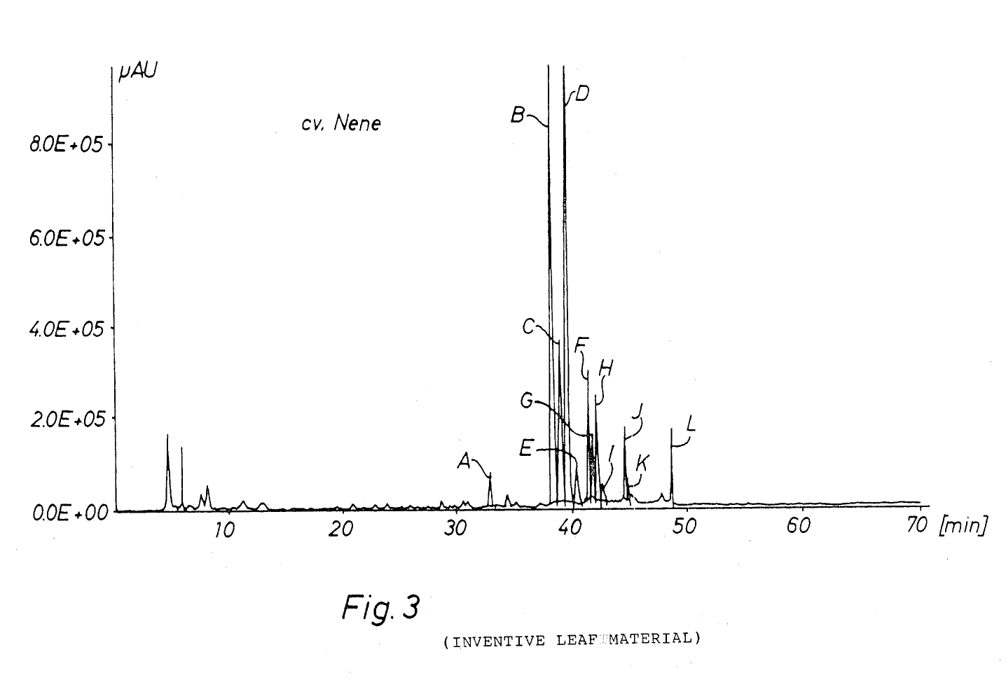

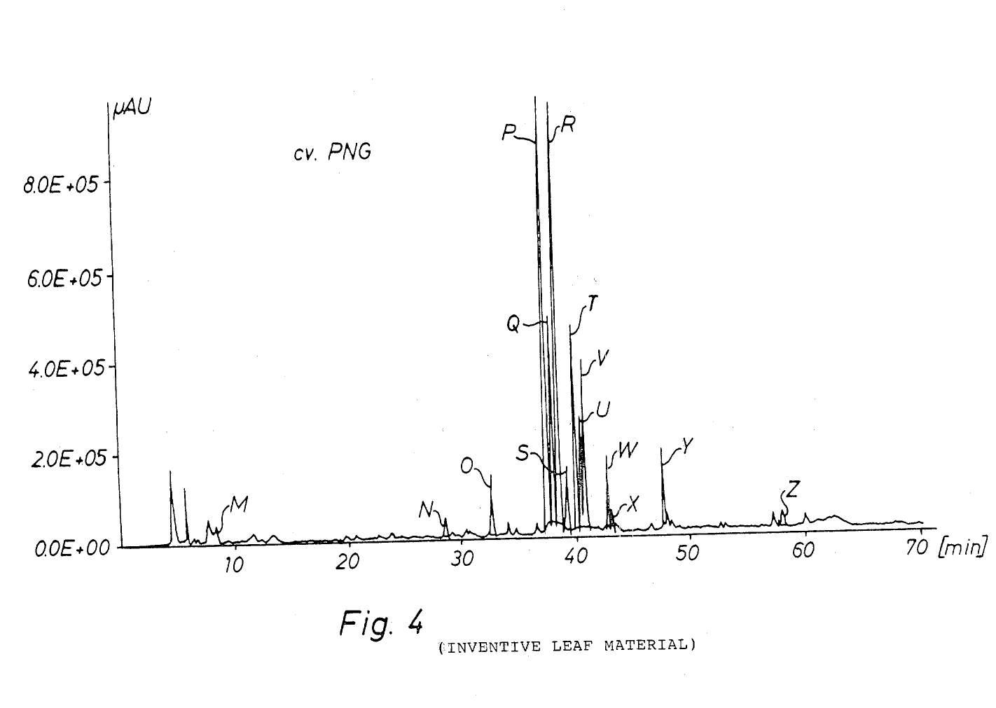

FIG. 3 shows the HPLC chromatogram of a further inventive extract of Piper methysticum G. Forster (Nene morphotype) from leaf material; and

FIG. 4 shows the HPLC chromatogram of a further inventive extract of Piper methysticum G. Forster (PNG morphotype) from leaf material.

The X axis of the HPLC chromatogram of FIGS. 1 to 4—as is common practice—indicates the retention time in minutes. The adsorption rate in micro units (μAU) was assigned to the Y axis.

The HPLC analysis for the chromatographic data given in FIGS. 1 and 2 was carried out as follows: 500 g of dried, pulverized leaves (for FIG. 1) or dried, pulverized rootstock of Piper methysticum G. Forster were twice extracted with 30 ml of methanol under 10 minutes of sonication. The extracts of each sample were then filtered and after evaporation of the solvent re-dissolved in 10 ml of methanol.

For HPLC analysis, an aliquot was filtered through a membrane of regenerated cellulose (pore diameter 0.45 μm). Then, an reversed phase HPLC was undertaken following the methodology described by Ross et al (cf . . . ) on a Spherisorb 5 ODS column (5 μm, 250×4.6 mm) using a Jasco HPLC system fitted with an auto sampler and diode array detector.

The samples were eluted with a mixture of 22 vol. % acetonitril, 18 vol. % methanol and 60 vol. % phosphoric acid (H₃PO₄) with a flow rate of 0.8 ml/minute at 60° C. The kavalactones were identified by comparing the retention times and the UV spectra with authentic standard samples.

The chromatograms of both extracts showed peaks identifiable as kavalactones. The leaf extract (FIG. 1) differed significantly from the root extract (FIG. 2). There was no methysticin in the leaf extract and there was a significant difference in the proportion of kavalactones in both extracts. There was a smaller proportion of kavain in the leaf extract, which is the primary kavalactone of the root extract. Dihydromethysticin and dihydrokavain were preponderantly in the leaf extract rather than the root extract. The leaf extract also showed a characteristic additional peak, which is probably significant, but utterly absent in the root extract.

The HPLC analysis for the data for FIGS. 2 and 3 diagrams was carried out as follows: 500 mg of dried, pulverized leaves from the Nene (FIG. 3) and PNG morphotype (FIG. 4) of Piper methysticum G. Forster were twice extracted with 30 ml of methanol under 10 minutes of sonication. The extracts of each sample were then filtered and after evaporation of the solvent re-dissolved in 10 ml of methanol.

For the HPLC analysis an aliquot was filtered through a membrane of regenerated cellulose (pore diameter 0.45 μm) and analysed with an HPLC system (Jasco) following Holzl and Ostrowski, i.e. with a diode array detector, a Hypersil 120-5 ODS (250×4.6 mm) column as a stationary phase and with a precolumn of the same material (both from Macheray-Nagel).

The mobile phase with a flow rate of 0.6 ml/min consisted of two solvent systems with (A) a mixture of 19 vol % acetonitril, 80 vol. % water and 1 vol. % H₃PO₄ and (B) a mixture of 59 vol. % acetonitril, 40 m vol. % methanol and 1 vol. % H₃PO₄, with a linear gradient (0-8 minutes 100 vol. % A, 8-30 minutes 50 vol. % A, 30-75 minutes 0 vol. % A). An auto sampler was used to inject 10 μl. Detection took place at UV/VIS 200-600 nm. Identification of the compounds at 253 nm was either implemented using external standard methods or using the UV spectra. The external standards used were rutosid, hyperosid, isoquercitrin, Quercetin (all from Roth, Germany), kaempferol (Sigma and Fluka, Switzerland), amentoflavon (Extrasynthese, France). Precision and selectivity were determined by analysis of the individual compounds and their mixtures.

The chromatograms of both extracts in FIGS. 3 and 4 match with regard to the main peaks showing various peaks in the more lipophilic area. No hydrophilic flavonoides, e.g. hyperosid, isoquercitrin, quercitrin and rutosid could be determined in the extracts of FIGS. 3 and 4.

The inventive extract of Piper methysticum G. Forster in accordance with a primary embodiment is displayed by an HPLC diagram which is essentially characterized by FIG. 1.

Its main features are as follows:

the extract is substantially free of methysticin (RT=14.8-15.9; typically 15.70), as evidenced by the HPLC diagram;

the HPLC diagram shows additionally a peak characteristic for dihydromethysticin at RT=15.8-16.7, typically 16.35 and a peak characteristic for dihydrokavain at RT=19.0-21.3, typically 20.14.

The peak characteristic for desmethoxyyangonin at RT=27.6-29.0, typically 28.69, and the peak characteristic for yangonin at RT=29.3-31.1, typically 30.25, are not particularly significant in the invention relevant extracts.

In general, the HPLC diagram further shows a peak at RT=25.0-25.9, typically 25.7.

The small peaks in FIG. 1 for RT values under 10 mins probably originate from the solvent and in this instance are not considered to be fingerprint characteristics.

A comparison of the HPLC chromatograms of the invention relevant extracts of leaf material with the HPLC chromatograms of known extracts of root material showed clear differences. The invention relevant methanol extracts obviously do not contain methysticin and also differ in the proportion of kavalactones. Whereas kavain is the main kavalactone in the root extract, the leaf extract contains only a small amount of it. In the leaf extract dihydromethysticin and dihydrokavain predominate. In addition the leaf extract shows a further peak at RT=25.0-25.9, typically 25.07, which is not present in the root extract.

As already stated, above-ground plant material of Piper methysticum G. Forster, preferably leaf material, is used to produce the invention relevant extract. It is advisable to use those morphotypes (varieties) of this plant which have been selected for high contents of active compounds.

The invention will now be explained in more detail with reference to receptor binding studies or binding affinities to receptors. Notably, the receptor binding studies show the increased pharmaceutical efficacy of extracts produced from leaf material of Piper methysticum G. Forster in comparison to extracts from root material of Piper methysticum G. Forster.

These comparative studies investigated the interactions of leaf and root extracts on selected receptors, particularly receptors affecting the central nervous system such as benzodiazepine, GABA_{A} (GABA binding site), dopamine D₂, serotonin (5-HT₆ and 5 HT₇), opioid (1 and 6), and histamine receptors (H₁ and H₂). The study used four morphotypes (cultivars or varieties) of Piper methysticum G. Forster, i.e. Mahakea, Nene, Purple Moi and PNG originating from the Hawaiian Islands.

Three year old Mahakea leaf material and Mahakea root material was obtained from the Wainani Farm in Hawaii. Nene, Purple Moi and PNG plants were propagated from stem material in a greenhouse in Witterswil, Switzerland (at 25±3° C., photoperiod: 16 h/day); the samples used in the study were collected from 18 months old plants.

To produce the methanol extracts for investigation, the leaves and root materials, particularly the lateral roots, were harvested and dried using a ventilation drier at 35° C. during a period of 48 hours. The dried powder was pulverized and then twice extracted in methanol in an ultrasonic bath for 15 minutes. After concentration of the solvent in the dryer, the residue was again taken up in methanol at a concentration of 50 mg/ml. The extract obtained was stored for later HPLC and the receptor studies at −20° C. A methanol concentration of 2% was not exceeded in the receptor studies.

Table 1 lists the percentage content of selected kavapyrones based on the dry weight of the experiment leaf and root materials of the four cultivars of Piper methysticum G. Forster as determined by the HPLC analysis. A Jasco HPLC system was utilized, coupled to a diode array detector (Jasco M D 910), the readings taken from an analytical Spheriscorb 5 ODS column (5 mm, 250×4.6 mm). The samples were eluted with a mixture of 22% acetonitril, 18% methanol and 60% H₃PO₄ (50 mM) using a flow rate of 0.8 ml/minute at 60° C. within 50 minutes. A standard kava extract obtained from Addipharma GmbH, Hamburg, Germany, EKP 001; Ch.B. 602140, was used for identification and calibration of the six main kavapyrones, i.e. kavain, 7,8-dihydrokavain, methysticin, 7,8-dihydromethysticin, yangonin and 5,6-desmethoxy yangonin. Yangonin and 7,8-dihydromethysticin were detected at a wavelength of 360 nm and the other four kavapyrones at a wavelength of 240 nm. The abbreviations used in table 1 are:

TABLE 1

| | DMY | Y | M | DHM | K | DHK | Total |
|---|---|---|---|---|---|---|---|
| Mahakea | | | | | | | |
| Leaf | 0.1 | 0.1 | 0.0 | 0.4 | 0.0 | 0.4 | 1.0 |
| Root | 1.1 | 1.7 | 2.0 | 1.1 | 1.9 | 1.3 | 9.1 |
| PNG | | | | | | | |
| Leaf | 0.1 | 0.2 | 0.0 | 2.2 | 0.1 | 1.8 | 4.4 |
| Root | 0.6 | 0.8 | 1.6 | 1.9 | 1.4 | 2.3 | 8.5 |
| Purple Moi | | | | | | | |
| Leaf | 0.1 | 0.1 | 0.0 | 1.1 | 0.1 | 1.1 | 2.4 |
| Root | 0.6 | 0.9 | 1.1 | 0.4 | 1.4 | 0.6 | 5.1 |
| Nene | | | | | | | |
| Leaf | 0.1 | 0.1 | 0.0 | 2.7 | 0.2 | 1.9 | 5.0 |
| Root | 1.0 | 0.7 | 1.5 | 0.9 | 1.1 | 0.8 | 5.9 |

K: Kavain
DHK: 7,8-dihydrokavain
M: Methysticin
DHM: 7,8-Dihydromethysticin
Y: Yangonin
DMY: 5,6-Desmethoxy-Yangonin
Total: Sum of K + DHK + M + DHM + Y + DMY

The HPLC diagrams of the preparations use the above annotations.

It is apparent from table I that the six main kavapyrones are present in similar quantities in the roots of the four cultivars of Piper methysticum G. Forster, whereby each of the six kavapyrones contributed between 10% and 20% to the total, i.e. to the total sum of the six kavapyrones. On the other hand, the HPLC analysis showed that two kavapyrones, DHK and DHM, are responsible for more than 70% of the total sum of kavapyrone in the leaf material in all the four cultivars of Piper methysticum G. Forster.

Only traces (<0.2%) of kavain could be detected in the leaf extracts, whereas the root extracts contained between 1.1% (Nene) and 1.9% (Mahakea) (percentage calculated based on the dry weight of the leaves and roots). Methysticin occurred at levels similar to kavain in the root extract, i.e. between 1% and 2%, but could not be detected in the leaf extracts. The percentage content of DHM and DHK was generally higher in the leaf extracts than in the root extracts. Exceptions are the Mahakea plants and the PNG plants in the case of DHK. The sum of the relative contents of the six kavapyrones based on the dry weight of the leaf extracts was: 2.4% for Purple Moi, 4.4% for PNG and 5.0% for Nene. The sum of the relative content of the six kavapyrones based on the dry weight of the root extracts was in the range 5.1% (for Purple Moi) to 9.1 (for Mahakea).

With the exception of benzodiazepine, GABA_{A} and dopamine D₂ receptors the receptors used in the study were obtained using the Semliki Forest virus expression system (abbreviated to SFV in the following). Benzodiazepine receptors were prepared from rat cortex, GABA_{A} receptors from rat cerebellum and dopamine D₂ receptors from calf striatum.

Expression of the receptors using the SFV system was undertaken in accordance with the method of U. Simmen, W. Burkard, K. Berger, W. Schaffner and K. Lundstrom as described in the Journal of Receptor and Transduction Research 19 (1999) 59-74. Human receptor cDNAs in pSFVI/pSFV2gen were subcloned using conventional biological procedures. To generate the virus particle, RNA with SP6RNA polymerase from the recombined receptor and pSFV helper2 carrying plasmids were transcribed and introduced into the cells of BHK (baby hamster kidney) using the electroporation method. After 24 hours the recombined virus particles were collected.

16-18 hours after infection, infected CHO (Chinese hamster ovary) cells were briefly washed in 5 nmM Hepes buffer pH 7.4, 2 mM EDTA and lysed in the same buffer for 20 minutes at 4° C. The lysed cells were then transferred to 10 ml centrifuge tubes, centrifuged at 40,000 g for 15 minutes and re-suspended in 50 mM Tris/HCl buffer pH 7.8, 1 mM EDTA and 5 mM MgCl₂ using a Polytron homogenisator. After centrifugation at 40,000 g for 15 minutes, the pellet was collected and stored at −80° C. until it was used for receptor binding studies (the other receptor material was also stored at −80° C.).

Rat brain (stem Wistar) obtained from Biological Research Laboratories Ltd, Füllingsdorf, Switzerland was used for the preparation of the GABA_{A} receptors. After isolating, the cerebellum was homogenized in a 1:50 volume of tris HCl buffer (50 mM Tris HCl, pH 7.4, 0.32 M sucrose, 1 mM EDTA, 0.02 NaN₃ and 0.1 mM PMSF) using a Polytron homogenizer for a period of 30 seconds and subsequently centrifuged at 500 g at 4° C. for 10 minutes The supernatant was then diluted in the 2 fold volume of the buffer and re-centrifuged at 4° C. at 18,000 g for 45 minutes. The resulting supernatant was discarded; the pellet was washed in buffer two times, whereby each time the suspension was centrifuged under the same conditions for 30 minutes and then the supernatant was discarded leaving the membrane pellet.

Preparation of the GABA_{A} was undertaken using rat brain of Wistar rats obtained from the Biological Research Laboratories Ltd., Füllingdorf, Switzerland. Once the cerebellum had been isolated it was homogenized in a 1:50 volume of Tris HCl buffer (50 mM Tris HCl, pH 7.4, 0.32 M sucrose, 1 mM EDTA, 0.02% NaN₃, and 0.1 mM PMSF) using a Polytron homogenisator for a period of 30 seconds and then centrifuged at 4° C. at 500 g for 10 minutes. The supernate was diluted with twice the volume of buffer and re-centrifuged at 4° C., 18,000 g for 45 minutes. The supernate was then discarded and the pellet washed twice with buffer, whereby each time the suspension was centrifuged under the same conditions for 30 minutes and then the supernate discarded leaving the membrane pellet.

Preparation of the benzodiazepine receptor was undertaken using rat brain cortex material. This was homogenized in a 1:40 volume of a Tris HCl buffers (15 mM Tris HCl, pH 7.4, 118 mM NaCl, 4.8 mM KCl, 1.2 mM CaCl₂, 1.2 mM MgCl₂) for 30 seconds. The suspension was then diluted in a 1:120 volume of buffer and centrifuged at 4° C., 18,000 g for 10 minutes. Once the supernatant had been decanted, the membrane pellet remained.

Preparation of the dopamine D₂ used calf striatum, which was homogenized in a 1:40 volume of a Tris HCl buffer (50 mM Tris HCl, pH 7.4, 0.1% ascorbic acid, 120 mM NaCl, 5 mM KCl, 2 mM CaCl₂, 1 mM MgCl₂) for 60 seconds. The homogenate was then centrifuged for 4° C., 18000 g for 10 minutes. Once the supernatant had been decanted, the membrane pellet remained.

In all cases, determination of the protein concentration was conducted using the BCA method in accordance with the report by P. K. Smith et al in Analytical Biochemistry 150 (1985), 76-85.

The receptor binding studies were replicated three times (experiment 1-3) in a total volume of 500 μl, under the conditions given in table 2. Binding equilibration was terminated by rapid filtration using a GF/C filter under reduced pressure and with a final triple wash with ice cooled 5 ml Tris/HCl pH 7.4 buffer. The radioactivity on the filter was determined by liquid scintillation analysis (Tri-Carb 2100 TR, Packard Bioscience Company). The IC₅₀ data were approximations determined from mathematical curves, which were based on individual measurements (P<0.01), i.e. the median±standard deviation.

TABLE 2
Receptors, radioligands and conditions of receptor binding studies

| | Species | Protein- | | Ligand- |
|---|---|---|---|---|
| Receptor | (Source) | conc.(μg) | Ligand | conc.(nM) |
| Opioid | | | | |
| μ | human (SFV- | 15-20 | ³H-Naloxone | 3.6 |
| | expression) | | (NEN) | |
| δ | human (SFV- | 15-20 | ³H-Deltorphin | 0.28 |
| | expression) | | (Amersham) | |
| Serotonin | | | | |
| 5-HT₆ | human (SFV- | 15-20 | ³H-LSD (NEN) | 1.2 |
| | Expression) | | | |
| 5-HT₇ | human (SFV- | 15-20 | ³H-LSD | 1.2 |
| | Expression) | | | |
| Histamine | | | | |
| H-1 | human (SFV- | 15-20 | ³H-Pyrilamine | 1.6 |
| | Expression) | | (Amersham) | |
| H-2 | human (SFV- | 15-20 | ³H-Tiotidine | 2.5 |
| | Expression) | | (NEN) | |
| GABA_{A} | rat | 15-20 | ³H-Muscimol | 2.0 |
| | (Cerebellum) | | (NEN) | |
| Benzodiazepine | rat | 200 | ³H-Flumazenile | 1.0 |
| | (Cortex) | | (Amersham) | |
| Dopamine D₂ | calf | 120 | ³H-Spiperone | 0.2 |
| | (Striatum) | | (Amersham) | |

Table 3 summarizes IC₅₀-values obtained from receptor binding studies of leaf and root extracts of the four Piper methysticum G. Forster cultivars. * Values represent means of triplicates from one to three experiments±standard error of the mean value.

TABLE 3
Effects of Piper methysticum Forster Extracts on Binding of Specific Radioligands to Selected CNS Receptors

| IC₅₀-values | | Dopamine | | Opioid | | Histamine | | Serotonin | |
|---|---|---|---|---|---|---|---|---|---|
| (μg/ml)* | Benzodiazepine | D₂ | GABA_{A} | μ | δ | H₁ | H₂ | 5-HT₆ | 5-HT₇ |
| Mahakea root extract | 860 ± 60 | 850 ± 22 | 87 ± 17 | 592 ± 34 | 185 ± 61 | 850 ± 37 | >1000 | >1000 | 492 ± 13 |
| Mahakea leaf extract | 510 ± 35 | 68 ± 4 | 4 ± 1 | 19 ± 5 | 240 ± 30 | 36 ± 7 | 4 ± 1 | >1000 | 127 ± 32 |
| PNG root extract | 556 ± 88 | 101 ± 32 | 83 ± 15 | 256 ± 69 | 168 ± 16 | 603 ± 64 | 630 ± 59 | >1000 | 472 ± 13 |
| PNG leaf extract | 710 ± 36 | 36 ± 18 | 1 ± 0.5 | 74 ± 11 | 161 ± 39 | 206 ± 33 | 215 ± 23 | >1000 | 338 ± 17 |
| Purple Moi root extract | 900 ± 97 | 374 ± 61 | 23 ± 4 | 980 ± 79 | 340 ± 32 | >1000 | >1000 | >1000 | 700 ± 34 |
| Purple Moi leaf extract | 860 ± 89 | 43 ± 16 | 6 ± 2 | 263 ± 42 | 71 ± 23 | 404 ± 91 | 240 ± 17 | >1000 | 395 ± 18 |
| Nene root extract | 830 ± 89 | 380 ± 82 | 5 ± 2 | 424 ± 16 | 390 ± 33 | >1000 | >1000 | >1000 | 905 ± 65 |
| Nene leaf extract | 490 ± 68 | 37 ± 8 | 3 ± 1 | 228 ± 22 | 134 ± 28 | 337 ± 23 | 374 ± 80 | >1000 | 326 ± 38 |

The most potent binding inhibition was observed for leaf extracts with regard to GABA_{A} receptors resulting in IC₅₀ values of approximately 3 μg/ml (IC₅₀: inhibitory concentration at which 50% of the specific binding is inhibited). The investigated root extracts were less inhibitory, here IC₅₀ values in the range of 5 μg/ml (Nene) to 87 (Mahakea) were determined. A very strong inhibition could also be observed for leaf extracts of Mahakea on the binding to the histamine H₂ receptor with an IC₅₀ value of approximately 4 μg/ml, whereas the root extract of Mahakea only showed an IC₅₀ value of approximately 806 μg/ml.

Similarly, the binding to dopamine D₂, opioid, serotonin (5-HT₇) and histamine receptors (H₁ and H₂) was more potently inhibited by the leaf extracts than by the root extracts of the Mahakea. Moderate to strong affinities of the leaf extracts were determined (I<IC₅₀ value<100 μg/ml), whereas the root extracts showed only weak activity with IC₅₀ values ranging from 100 μg/ml to higher than 1000 μg/ml.

Large differences in binding inhibition were observed between the various cultivars. The most potent inhibition detected was found for the histamine receptors (H₁ and H₂) caused by the leaf extract of the Mahakea and the weakest inhibition caused by the root extract of Purple Moi and Nene. Varying affinities were also found for the binding to the opioid receptors, here the strongest affinity was found for the P-opioid receptors caused by the leaf extract from Mahakea (IC₅₀=19±5 μg/ml) and the weakest affinity caused by the leaf extract of the Purple Moi (IC₅₀=263±42) μg/ml. In the case of the μ-opioid receptor, the strongest affinity was found for the leaf extract of the PNG (IC₅₀=71±23 μg/ml) The binding to the benzodiazepine and to serotonin receptors (5-HT₆ and 5-HT₇) was only weakly inhibited by the extract of Piper methysticum G. Forster. The IC₅₀ values for the benzodiazepine receptors were 500 μg/ml and higher, for serotonin 5-HT₆ receptors even higher than 1000 μg/ml. For serotonin 5-HT₆ receptors the IC₅₀ values for the leaf extract were between 127 μg/ml (Mahakea) and 395 μg/ml (Purple Moi).

The binding studies using selected receptors occurring in the central nervous system showed the unexpected in vitro pharmacological action of leaf extracts of Piper methysticum G. Forster in comparison with the root extracts. Since the content of the six kavapyrones (considered to be the active constituents) is lower in the leaf extracts than in the root extracts of Piper methysticum G. Forster (calculation based on dry weight), these pharmacological results obtained from the binding studies are surprising and interesting. The non-existing correlation between the pharmacological potency of the extracts (as determined by the receptor binding studies) and the kavapyrone content of these extracts (determined by HPLC measurements) is surprising, since kavapyrones generally are held responsible for the pharmacological efficacy of Kava extracts; the result suggests the presence of additional pharmacologically active substances occurring in the leaf extracts.

The exact numeric values of the HPLC chromatograms shown in FIGS. 1-4 are given below:

| | | | | |
|---|---|---|---|---|
| Peak No. | Substance | RT | Peak area | RT-Range |

Figure 1:
Leaf extract

| | | | | |
|---|---|---|---|---|
| Peak 1 | Methysticin | missing | 0 | nd |
| Peak 2 | Dihydromethysticin | 16.35 | 17914 | 15.8-16.7 |
| Peak 3 | Davain | 18.43 | 3582 | 17.5-18.9 |
| Peak 4 | Dihydrokavain | 20.14 | 14776 | 19.0-21.3 |
| Peak 5 | Unidentified | 25.07 | 3655 | 25.0-25.9 |
| Peak 6 | Desmethoxyyangonin | 28.69 | 384 | 27.6-29.0 |
| Peak 7 | Yangonin | 30.25 | 854 | 29.3-31.1 |

Figure 2:
Root extract

| | | | | |
|---|---|---|---|---|
| Peak 1 | Methysticin | 15.70 | 14527 | 14.8-15.9 |
| Peak 2 | Dihydromethysticin | 16.37 | 6124 | 16.1-17.2 |
| Peak 3 | Davain | 18.40 | 22923 | 17.3-19.1 |
| Peak 4 | Dihydrokavain | 20.17 | 6592 | 19.2-20.5 |
| Peak 5 | Unidentified | missing | 0 | nd |
| Peak 6 | Desmethoxyyangonin | 28.65 | 5651 | 27.3-29.6 |
| Peak 7 | Yangonin | 30.24 | 5112 | 29.3-31.3 |

Figure 3:

| Description | RT | Area [μAU.sec] |
|---|---|---|
| A | 32.730 | 921223.500 |
| B | 38.177 | 15648714.336 |
| C | 38.813 | 4784182.200 |
| D | 39.477 | 15473329.942 |
| E | 40.240 | 1017960.516 |
| F | 41.250 | 2795177.600 |
| G | 41.627 | 1364085.585 |
| H | 41.983 | 1954062.604 |
| I | 42.467 | 463513.879 |
| J | 44.433 | 1456578.693 |
| K | 44.727 | 283436.785 |
| L | 48.463 | 870611.600 |

Total peak area = 47032877.240 [μAU.sec]
Figure 4:

| Description | RT | Area [μAU.sec] |
|---|---|---|
| M | 8.410 | 387891.479 |
| N | 28.547 | 509802.186 |
| O | 32.567 | 1449506.466 |
| P | 37.413 | 15132578.200 |
| Q | 37.880 | 5310814.000 |
| R | 38.443 | 14880170.730 |
| S | 39.117 | 1328780.200 |
| T | 39.940 | 4019500.540 |
| U | 40.333 | 2180868.169 |
| V | 40.637 | 3920823.431 |
| W | 42.673 | 1514505.232 |
| X | 42.983 | 242147.510 |
| Y | 47.533 | 1295667.212 |
| Z | 57.837 | 469412.619 |

Total peak area = 52642467.974 [μAU.sec]

1. An extract from Piper methysticum G. Forster, which extract is characterized by an HPLC diagram substantially having the features of FIG. 1.

2. The extract of claim 1, characterised in that it is substantially free of methysticin (RT=14.8-15.9, notably 15.70) as evidenced by said HPLC diagram.

3. The extract of claim 1 or 2, characterised in that said HPLC diagram has a peak at RT=15.8-16.7, notably 16.35, which is characteristic for dihydromethysticin.

4. The extract of any of claims 1-3, characterised in that said HPLC-diagram has a peak at RT=19.0-21.3, notably at 20.14, characteristic for dihydrokavain.

5. The extract of any of claims 1-6, characterised in that said HPLC diagram has a peak at RT=25.0-25.9, notably at 25.7.

6. An extract from Piper methysticum G. Forster, characterised in that it has been obtained by extraction of aboveground-growing parts of Piper methysticum G. Forster, notably leaf material, by means of an extracting agent.

7. The extract of claim 6, characterised by having essentially the features of FIG. 3 or 4.

8. A method of producing an extract according to any of claims 1-7, characterised in that one recovers said extract from aboveground-growing parts of Piper methysticum G. Forster, preferably the leaf material, preferably by extration of said plant material with a lower alkanol, notably and preferably methanol or ethanol, optionally in mixture with another organic solvent or with water; or with carbon dioxide in a liquid or supercritical state, optionally in mixture with a lower alkanol.

9. A use of an extract according to any of claims 1-7 for production of a pharmaceutical product that has an anxiolytic, anticonvulsive, muscle relaxant, narcopromotive, analgetic, sleep inducing, anti-phlogistic and/or neuro-protective effect.

10. A pharmaceutical product having an anxiolytic, anticonvulsive, muscle relaxant, narcopromotive, analgetic, sleep inducing, antiphlogistic and/or neuroprotective effect, characterised in that it consists, at least in part, of an extract which is obtained by extraction of aboveground-growing parts of Piper methysticum G. Forster, notably the leaf material, by means of an extracting agent.
